(19) United States
(12) Patent Application Publication
(10) Pub. No.: US 2001/0033844 A1
(43) Pub. Date: Oct. 25, 2001

(54) Human Herpesvirus 6A and 6B transfer factors for the treatment of chronic fatigue syndrome and multiple sclerosis

(76) Inventors: Gregory Bruce Wilson, Greenville, SC (US); R. Riley Shuler, Camerson, SC (US)

Correspondence Address: Cooper & Dunham LLP, 1185 Avenue of the Americas, New York, NY, 10036, US

(21) Appl. No.: 09/776,010

(22) Filed: Feb. 2, 2001

Related U.S. Application Data
(60) Provisional application No. 60/179,647, filed on Feb. 2, 2000.

(51) Int. Cl. A61K 39/245

(52) U.S. Cl. 424/229.1; 424/439

(57) ABSTRACT

The present invention provides transfer factors that confer cell-mediated immunity to Human Herpesvirus-6A and Human Herpesvirus-6B. The invention also provides pharmaceutical compositions comprising the transfer factors and methods of treating abnormalities in a subject using the transfer factors.

This application claims the benefit of copending U.S. Provisional Application No. 60/179,647, filed Feb. 2, 2000, the contents of which are hereby incorporated by reference.

### BACKGROUND

Throughout this application, various references are referred to within parentheses. Disclosures of these publications in their entireties are hereby incorporated by reference into this application to more fully describe the state of the art to which this invention pertains. Full bibliographic citation for these references may be found at the end of this application, preceding the claims.

The present invention relates to the development of transfer factors (TF) specific for human Herpesvirus 6A and 6B for the treatment of human patients clinically diagnosed with either chronic fatigue syndrome (CFS) (1) or multiple sclerosis (MS) (2).

Recent scientific studies provide evidence for the possible role of active infection with human Herpesvirus-6 (HHV-6) in MS and CFS (3, 4, 5, 6, 7, 8). The HHV-6 virus infects several cells of the immune system (CD4, CD8, NK cells) and is also neurotropic (9, 10, 11). HHV-6 is clearly immune suppressive, affecting cell-mediated immunity (CMI) and natural killer (NK) cell function (12, 13, 14, and 15). HHV-6 viral induced immune suppression (of which a profound defect of natural killer cell function is the most consistent finding) may allow for recurring reactivation of HHV-6 and resultant chronic active HHV-6 infection in patients with CFS and MS (5, 14, and 16).

It is known that TF prepared from lymphocytes or from colostrum from immune donor animals can be used to stimulate or transfer CMI against certain disease causing agents in man and other animals and that this transfer of CMI can be made between species (17, 18). No one has reported attempting to use TF specific for HHV-6A or HHV-6B from any source for the treatment of patients with MS. Attempts to treat CFS patients with TF have been reported (19,20,21). However, either TF of unknown specificity was used (19) or mixed preparations containing TFs against cytomegalovirus (CMV) and Epstein-Barr virus (EBV) as well as HHV-6 were used (20,21) and it was not clarified if the TF donors were immune to HHV-6A or HHV-6B. In addition, when preparations containing TFs for CMV, EBV and HHV-6 were compared to TF preparations containing just TFs for CMV and EBV for their effects on CFS patients, the results were equivocal (20) and no more effective than using TF preparations of unknown specificity (19). From prior studies it was therefore not clear that TFs specific for HHV-6A and HHV-6B could be used to treat CFS or MS patients.

### SUMMARY OF THE INVENTION

The present invention provides a transfer factor wherein the transfer factor confers cell-mediated immunity to Human Herpesvirus-6.

The invention provides a method of enhancing an immune response to Human Herpesvirus-6 in a subject, which comprises applying to the subject an amount of any of the transfer factors described herein effective to enhance the immune response of the subject to Human Herpesvirus 6.

The invention provides a method of treating Chronic Fatigue Syndrome in a subject, which comprises administering to the subject an amount of any of the transfer factors described herein effective to treat the Chronic Fatigue Syndrome.

The invention provides a method of treating Multiple Sclerosis in a subject, which comprises administering to the subject an amount of any of the transfer factors described herein effective to treat the Multiple Sclerosis.

The invention provides a method of treating an abnormality in a subject, which comprises administering to the subject an amount of any of the transfer factors described herein effective to alleviate the abnormality, wherein the abnormality is alleviated by enhancing the immune response to Human Herpesvirus-6.

The invention provides a pharmaceutical composition comprising any of the transfer factors described herein and a pharmaceutically acceptable carrier.

The invention provides the use of any of the transfer factor described herein for the preparation of a pharmaceutical composition for treating an abnormality, wherein the abnormality is alleviated by enhancing the immune response to Human Herpesvirus-6.

The invention provides an edible composition comprising any of the transfer factors described herein and an edible carrier. The invention provides the use of any of the transfer factor described herein for the preparation of an edible composition for treating an abnormality, wherein the abnormality is alleviated by enhancing the immune response to Human Herpesvirus-6.

### DETAILED DESCRIPTION OF THE INVENTION

The following definitions are presented as an aid in understanding this invention:

CFS—Chronic Fatigue Syndrome,

CMI—cell-mediated immunity,

CMV—Cytomegalovirus,

DTH—delayed type hypersensitivity,

EBV—Epstein-Barr virus,

HHV—Human Herpesvirus,

HHV-GA—Human Herpesvirus-6A,

HHV-6B—Human Herpesvirus-6B,

IBR—Infectious Bovine Rhinotracheitis virus,

LMI—leukocyte migration inhibition,

MS—Multiple Sclerosis,

NK—Natural Killer,

TF—Transfer Factor.

Having due regard to the preceding definitions, the present invention provides a transfer factor wherein the transfer factor confers cell-mediated immunity to Human Herpesvirus-6. In one embodiment of the invention the transfer factor confers cell-mediated immunity to Human Herpesvirus-6A. In another embodiment of the invention the transfer factor confers cell-mediated immunity to Human Herpesvirus-6B. In another embodiment of the invention the transfer factor confers cell-mediated immunity to both Human Herpesvirus-6A and Human Herpesvirus-6B.

The present invention provides a method of producing the transfer factors disclosed herein which comprises immunizing a lactating animal with Human Herpesvirus-6A or Human Herpesvirus-6B or both Human Herpesvirus-6A and 6B, recovering colostrum from the animal, and preparing the transfer factor from the colostrum. In one embodiment the transfer factor is produced by obtaining a cell-free fluid containing excreted transfer factor specific for Human Herpesvirus-6A and 6B which comprises collecting material secreted by the mammary gland of a suitable lactating mammal, treating the material to separate cells, cell debris, casein, fat and other substances which interface with transfer factor efficacy so as to produce a cell-free fluid containing the excreted transfer factor, discarding the separated cells, cell debris, casein, fat and other substances, and recovering the cell-free fluid containing the excreted transfer factor. (Method of producing transfer factors from colostrum detailed in U.S. Pat. No. 4,816,563.) In one embodiment the lactating animal is a bovid.

The present invention provides a method of producing the transfer factors disclosed herein which comprises immunizing an animal with Human Herpesvirus-6A or 6B or both Human Herpesvirus-6A and 6B, recovering an immune system component from the animal and preparing transfer factor specific for Human Herpesvirus-6A or 6B, or both for Human Herpesvirus-6A and 6B from a component of the immunized animal's immune system. In one embodiment the immune system component is dialyzable leukocyte extract. In another embodiment the immune system component is immune organ lysate such as spleen and lymph nodes. In another embodiment the immune system component is lymphoblastoid cells derived from the immune system of the immunized animal. In another embodiment the immune system component is a cell lines derived from the immune system of the immunized animal. Transfer Factor preparation using standard methods is more fully described in Fudenberg and Pizza (17).

The invention provides a method of producing a composition comprising any of the transfer factors described herein and a carrier. In one embodiment the composition is a pharmaceutical composition and the carrier is a pharmaceutically acceptable carrier. In another embodiment the compostion is an edible compostion and the carrier is an edible carrier.

In the subject invention, a “pharmaceutically effective amount” is any amount of a compound which, when administered to a subject suffering from a disease against which the compound is effective, causes reduction, remission, or regression of the disease. Furthermore, as used herein, the phrase “pharmaceutically acceptable carrier” means any of the standard pharmaceutically acceptable carriers. Examples include, but are not limited to, microcrystalline cellulose, rice powder, phosphate buffered saline, physiological saline, water, and emulsions, such as oil/water emulsions.

The invention provides a method of treating a subject's disease, which comprises applying to the subject an amount of any of the transfer factors described herein effective to treat the disease. In one embodiment, the disease is Chronic Fatigue Syndrome. In another embodiment the disease is Multiple Sclerosis. In one embodiment, the transfer factor enhances the subject's immune response to Human Herpesvirus-6A. In another embodiment, the transfer factor enhances the subject's immune response to Human Herpesvirus-6B. In another embodiment the transfer factor enhances the subject's immune response to both Human Herpesvirus-6A and Human Herpesvirus-6B.

The invention provides a method of treating an abnormality in a subject, which comprises administering to the subject an amount of any of the transfers factors described herein effective to alleviate the abnormality, wherein the abnormality is alleviated by enhancing the subject's immune response to Human Herpesvirus-6. In one embodiment, the transfer factor enhances the subject's immune response to Human Herpesvirus-6A. In another embodiment, the transfer factor enhances the subject's immune response to Human Herpesvirus-6B. In another embodiment the transfer factor enhances the subject's immune response to both Human Herpesvirus-6A and Human Herpesvirus-6B.

The invention provides the use of any of the transfer factors described herein for the preparation of a pharmaceutical composition for treating an abnormality, wherein the abnormality is alleviated by enhancing the subject's immune response to Human Herpesvirus-6. In one embodiment, the transfer factor enhances the subject's immune response to Human Herpesvirus-6A. In another embodiment, the transfer factor enhances the subject's immune response to Human Herpesvirus-6B. In another embodiment the transfer factor enhances the subject's immune response to both Human Herpesvirus-6A and Human Herpesvirus-6B.

The invention provides the use of any of the transfer factors described herein for the preparation of an edible composition for treating an abnormality, wherein the abnormality is alleviated by enhancing the subject's immune response to Human Herpesvirus-6. In one embodiment, the transfer factor enhances the subject's immune response to Human Herpesvirus-6A. In another embodiment, the transfer factor enhances the subject's immune response to Human Herpesvirus-6B. In another embodiment the transfer factor enhances the subject's immune response to both Human Herpesvirus-6A and Human Herpesvirus-6B.

The following Experimental Details are set forth to aid in an understanding of the invention, and are not intended, and should not be construed, to limit in any way the invention set forth in the claims which follow thereafter.

Experimental Details

In the present invention, TFs able to induce CMI to both known types of HHV-6 (HHV-6A and HHV-6B) were prepared and used for the first time as a treatment modality for active HHV-6 infection and the associated immune suppression in human patients with CFS or MS. Positive results were obtained in the majority of CFS patients administered the HHV-6A and HHV-6B TF preparations as evidenced by (a) increased NK cell function and (b) decreased clinical symptom scores. Positive results were also obtained in the majority of MS patients administered the HHV-6A and HHV-6B TF preparations as evidenced by (a) increased NK cell function and (b) prevention of worsening of clinical symptoms. Suitable control preparations that lacked TFs for HHV-6A and HHV-6B failed to increase NK cell function or to affect the clinical symptoms of either CFS or MS patients.

In the present invention, colostrum samples from bovines immunized with HHV-6A and HHV-6B antigens were used as the source for the preparation of TF. In practice, however, another source of TF could be used provided the TF donor was first made immune with HHV-6A and HHV-6B antigens, either by immunization with an antigen or injection with TF, since TF's effectiveness may not depend upon the species of the donor per se. In the present invention the TF was administered orally, however, another route of administration of the TF could be used for example subcutaneously or intramuscularly. In the present invention, the potency units of TFs for HHV-6A and HHV-6B were determined and certification of the TF potency was considered important to expedite determining the dosage of HHV-6A and HHV-6B TFs the CFS and MS patients needed to receive to provide immunological and clinical benefit.

Results

Experiments were initially performed to determine the amount of TF patients should receive and the frequency of administration of the TF. Based upon these results a placebo controlled double blind experiment was performed consisting of two patient groups. Group I (HHV-6 TF Group) consisted of CFS and MS patients who received capsules containing the HHV-6A and HHV-6B TF. Group II (Placebo TF Group) consisted of CFS and MS patients who received capsules of a control TF preparation not containing HHV-6A or HHV-6B TF but containing an equivalent amount of Infectious Bovine Rhinotracheitis virus transfer factor (IBR-TF) based upon potency units. Both Groups were evaluated over a period of four months using the criteria described under “Patients Studied” in the Materials and Methods section. All patients received 2 capsules three times a day during day 1 to 5 (start of month 1), day 31 to 35 (start of month 2) and day 61 to 65 (start or month 3) of the study. Each capsule contained 20 potency units of HHV-6A and HHV-6B TF or an equivalent amount of IBR-TF (Placebo control). The capsules were taken orally with water prior to eating.

Tables 2, 3 and 4 present a summary of the results obtained from the double-blind study.

As shown in Table 2, 5 of 8 CFS patients who received the HHV-6 TF had a 50% or greater reduction in their symptom score and 5 of 8 CFS patients showed an increase of 50% or greater in their NK cell function. Only 2 MS patients received the HHV-6 TF during this phase of our study (Table 3; HHV-6 Intermittent ). Neither MS patient had a worsening of clinical symptoms and one of them showed a 50% or greater increase in NK cell function (Table 3). In contrast, zero of 10 CFS patients who received the placebo TF showed a 50% or greater reduction in their symptom score and zero of 10 CFS patients who received the placebo TF showed a 50% or greater increase in NK cell function (Table 4). Only two MS patients received the placebo control TF (Table 3; Placebo Intermittent ). Neither MS patient had a 50% or greater increase in NK cell function and one of the two patients had a worsening of MS symptoms during the course of the study (Table 3).

Following the completion of the double blind study, we continued to investigate the amount of HHV-6A and HHV-6B TF to give each patient and the frequency of dosing to achieve maximum benefit. This application discloses a dosage regimen that is effective in the majority of CFS and MS patients. Tables 3 and 5 present results we have obtained for 10 CFS patients (Table 5) and 5 MS patients (Table 3; HHV-6 Daily ) who received a daily dose of 80 or 120 potency units of HHV-6A and HHV-6B TF over a course of three months. Nine of 10 CFS patients showed a decrease in their symptom score of 50% or greater and 9 of 10 CFS patients showed a 50% or greater increase in NK cell function (Table 5). None of five MS patients showed a worsening of clinical symptoms and four of five MS patients showed a 50% or greater increase in NK cell function during the three month study period.

Conclusions

1. This application discloses TFs able to induce CMI to both known types of HHV-6, HHV-6A and HHV-6B, and their use for the first time as a treatment modality for active HHV-6 infection and associated immune suppression in human patients with CFS or MS.

2. The majority of CFS patients and MS patients showed an increase in NK cell function of 50% or greater indicating a positive benefit to their cellular immune function as a result of receiving HHV-6A and HHV-6B TF.

3.The majority of CFS and MS patients obtained clinical benefit as a result of receiving HHV-6A and HHV-6B TF as evidenced by a 50% or greater decrease in their symptom score (CFS patients) or by no worsening of their symptoms (MS patients).

4. The effectiveness of the HHV-6A and HHV-6B TF in treating both CFS and MS patients depends upon the use of TF preparations of known potency and careful optimization of the dosage regimen.

Materials and Methods

Preparation of Transfer Factor:

Colostrum from dairy cows was used as a source of both HHV-6A and HHV-6B TF and Control TF preparations (Placebo or Control TF preparations). (For general method of producing transfer factors from colostrum see U.S. Pat. No. 4,816,563.) To obtain HHV-6A and HHV-6B TF, pregnant dairy cows were immunized prior to calving with HHV-6A or HHV-6B viral antigens. Separate cows were injected with either HHV-6A or HHV-6B viral antigens. Cows not immunized with HHV-6A or HHV-6B viral preparations were used to obtain colostrum for control TF preparations. All cows were also immunized with commercially IBR virus vaccine following the directions supplied by the manufacturer. TF rich fractions were obtained from post-parturition colostrum following the methods of Wilson and Paddock (18). The HHV-6A, HHV-6B, and suitable control TF-rich fractions were lyophilyzed and stored dry at −20 C or lower until used. When the amount of TF to use for treating CFS and MS patients was determined (as noted in the next section), the TF powder was mixed with an inert filler (microcrystalline cellulose) and incorporated into gelatin capsules.

Testing of TF Preparations for TF Activity and Potency:

The presence of HHV-6A TF, HHV-6B TF or IBR TF was determined using a delayed-type hypersensitivity (DTH;footpad swelling) assay in mice as described by Rifkind et al. (22) and Petersen et al. (23). For each type of TF evaluated, the mouse DTH assay parameters were set-up such that 5 potency units of TF as measured in vitro using the leukocyte migration inhibition (LMI) assay would produce significant DTH in mice. The LMI assay has been used historically as a tool for determining potency units of TF preparations to be used for immunotherapy and immunoprophylaxis (24).

Patients Studied:

The patients evaluated in this study had a confirmed diagnosis of either CFS or MS using established criteria for CFS and for MS (1,2). Patient symptoms were scored using 32 parameters as noted in the Symptom Profile sheet shown as Table 1. HHV-6 viral blood cultures were performed by the Wisconsin Viral Research Group utilizing a rapid viral blood culture method developed by their group (5). Culture results were reported as positive or negative. Natural killer (NK) cell function assays were performed as described by Bryant et al. (25) and are reported as lytic units.

All patients were evaluated for clinical symptoms (symptom score), HHV-6 viral blood culture status and NK cell function prior to the initiation of TF treatment and at least every four weeks during treatment for a period of up to six months.

TABLE 1
Symptoms Profile Sheet

| Symptom | Intensity (0-4) | Comment |
|---|---|---|
| Fatigue/Exhaustion | | |
| Increase Sleep Required | | |
| Sleep Disturbance | | |
| Awake Unrested | | |
| Decreased Activity Level | | |
| Worse After Activity | | |
| Fever | | |
| Night Sweats | | |
| Sore Throat | | |
| Lymph Gland Tender/Swelling | | |
| Headache | | |
| Neck/Back Ache | | |
| Muscle Ache | | |
| Joint Ache | | |
| Weakness Generalized | | |
| Dizziness/Light-headed | | |
| Nausea/Vomiting | | |
| Diarrhea | | |
| Anxiety | | |
| Mood Swings | | |
| Depression | | |
| Numbness/Tingling | | |
| Weakness Localized (arm, leg, etc) | | |
| Muscle Spasm/Twitching | | |
| Tremor | | |
| Imbalance | | |
| Visual problems (ex. Focusing) | | |
| Light sensitive | | |
| Memory problems | | |
| Concentration problems | | |
| Attention Span Problems | | |
| Confusion | | |
| OTHER | | |
| 1 | | |
| 2 | | |
| 3 | | |
| 4 | | |
| 5 | | |
| Activity Daily Living | Check one | |
| Bedridden (do virtually nothing) | | |
| Shut-in (can't do even light work) | | |
| Partial (can do part-time work) | | |
| Full-time limited (work full time) | | |
| Full-time unlimited (normal function) | | |

Intensity scale: 0 - not present, 1 - present but mild, 2 - moderate, 3 - present a lot of the time, 4 - almost always present

TABLE 2
Results of Double Blind Study of CFS Patients (HHV-6 TF Group)

| | | | | Symptom score | NK Function | HHV-6 positive |
|---|---|---|---|---|---|---|
| Patient | Age | Sex | Diagnosis | decrease 50% or > | Increase 50% or > | culture on RX |
| 1 | 45 | F | CFS | No | No | Yes |
| 2 | 44 | M | CFS | Yes | Yes | Yes |
| 3 | 38 | F | CFS | Yes | No | No |
| 4 | 57 | F | CFS | No | Yes | No |
| 5 | 43 | M | CFS | Yes | Yes | Yes |
| 6 | 59 | F | CFS | Yes | Yes | Yes |
| 7 | 40 | F | CFS | Yes | Yes | Yes |
| 8 | 31 | F | CFS | No | No | Yes |
| Total | | | | 5 | 5 | 6 |

TABLE 3
Results for Treatment of MS Patients with Transfer Factor

| | | | | Symptom | NK Function | HHV-6 positive | |
|---|---|---|---|---|---|---|---|
| Patient | Age | Sex | Diagnosis | worsening | increase 50% or > | culture on RX | TF Group |
| 1 | 41 | F | MS | No | Yes | Yes | HHV-6 Daily |
| 2 | 31 | M | MS | No | Yes | Yes | HHV-6 Daily |
| 3 | 36 | F | MS | No | Yes | Yes | HHV-6 Daily |
| 4 | 60 | F | MS | No | No | Yes | HHV-6 Daily |
| 5 | 51 | F | MS | No | Yes | No | HHV-6 Daily |
| 6 | 42 | F | MS | No | No | No | HHV-6 Intermittent |
| 7 | 43 | F | MS | No | Yes | Yes | HHV-6 Intermittent |
| 8 | 37 | F | MS | No | No | No | Placebo Intermittent |
| 9 | 49 | F | MS | Yes | No | Yes | Placebo Intermittent |

TABLE 4
Results of Double Blind Study of CFS Patients (Placebo TF Group)

| | | | | Symptom score | NK Function | HHV-6 positive |
|---|---|---|---|---|---|---|
| Patient | Age | Sex | Diagnosis | decrease 50% or > | Increase 50% or > | culture on RX |
| 1 | 38 | F | CFS | No | No | Yes |
| 2 | 56 | F | CFS | No | No | Yes |
| 3 | 51 | F | CFS | No | No | Yes |
| 4 | 58 | M | CFS | No | No | Yes |
| 5 | 56 | F | CFS | No | No | No |
| 6 | 56 | M | CFS | No | No | Yes |
| 7 | 56 | F | CFS | No | No | No |
| 8 | 43 | F | CFS | No | No | Yes |
| 9 | 36 | F | CFS | No | No | Yes |
| 10 | 52 | F | CFS | No | No | Yes |
| Totals | | | | 0 | 0 | 8 |

TABLE 5
Results of Daily Dosing of CFS Patients with HHV-6 TF

| | | | | Symptom score | NK Function | HHV-6 positive |
|---|---|---|---|---|---|---|
| Patient | Age | Sex | Diagnosis | decrease 50% or > | Increase 50% or > | culture on RX |
| 1 | 37 | F | CFS | Yes | Yes | No |
| 2 | 47 | M | CFS | Yes | Yes | No |
| 3 | 50 | F | CFS | Yes | Yes | Yes |
| 4 | 49 | F | CFS | Yes | Yes | No |
| 5 | 48 | F | CFS | No | Yes | Yes |
| 6 | 41 | F | CFS | Yes | Yes | No |
| 7 | 45 | F | CFS | Yes | No | No |
| 8 | 34 | F | CFS | Yes | Yes | Yes |
| 9 | 57 | F | CFS | Yes | Yes | No |
| 10 | 38 | M | CFS | Yes | Yes | Yes |
| Totals | | | | 9 | 9 | 4 |

### REFERENCES

1. Fikuda K, Strauss S E, Hickie I et al. The chronic fatigue syndrome: A comprehensive approach to its definition and study. Ann Intern Med 1994; 121:953-959.

2. Rudick R A, Cohen J A, Weinstock-Guttman B et al. Management of multiple Sclerosis. N. Engl. J Med 1997; 337:1604-1611.

3. Challoner P B, Smith K T, Parker J D et al. Plaque associated expression of human Herpesvirus-6 in multiple sclerosis. Proc Natl Acad Sci 1995;92:7440-7444.

4. Carrigan D R, Harrington D and Knox K K. Subacute leukoencephalitis caused by CNS infection with human Herpesvirus six manifesting as acute multiple sclerosis. Neurology 1996;47:145-148.

5. Brewer J H, Knox and Carrigan D R. Active human Herpesvirus-6 infections are present in the CNS, lymphoid tissues and peripheral blood of patients with multiple sclerosis. Abstract 57. IDSA 36^{th} Annual Meeting. Nov. 12-15, 1998. Denver, Colo.

6. Buchwald D, Cheney P R, Peterson D L et al. A chronic illness characterized by fatigue, neurologic and immunologic disorders, and active human Herpesvirus type 6 infection. Ann Intern Med 1992;116:103-113.

7. Zorenzenon M, Rukh G Botta G A et al. Active HHV-6 infection in chronic fatigue syndrome patients from Italy: New data. J Chron Fatigue Syndr 1996;2(4):3-12.

8. Knox K K, Brewer J H, and Carrigan D R. Persistent active human Herpesvirus six (HHV-6) infections in patients with chronic fatigue syndrome. J Chron Fatigue Syndr 1999;5:245-246.

9. Lusso P, Malnati M, De Maria A et al. Productive infection of CD4+ and CD8+ mature human T cell populations and clones by human Herpesvirus 6. J Clin Microbiol 1991;147:685-691.

10. Lusso P, Malnati M, Garzino-Demo A et al. Infection of natural killer cells by human Herpesvirus 6. Nature 1993;362:458-462.

11. Caserta M T, Hall C B, Schnabel K et al. Neuroinvasion and persistence of human Herpesvirus-6 in children. J Infect Dis 1994;170:1585-1589.

12. Klimas N G, Salvato F, Morgan R et al. Immunologic abnormalities in chronic fatigue syndrome. J Clin Microbiol 1990;28;1403-1410.

13. Whiteside T L and Friberg D. Natural killer cells and natural killer cell activity in chronic fatigue syndrome. Am J Med 1998;105:27S-34S.

14. Brewer J H, Knox K K, and Carrigan D R. Severe dysfunction of natural killer (NK) cells associated with chronic active human Herpesvirus-6 (HHV-6) viremia in patients with chronic fatigue syndrome. Abstract. IDSA. 37^{th} Annual Meeting. Nov. 18-21, 1999. Philadelphia, Pa.

15. Kastrukoff L K, Morgan N G, Zecchini D et al. A role for natural killer cells in the immunopathogenesis of multiple sclerosis. J Neuroimmunol 1998;86:123-133.

16. Brewer J H, Knox K K and Carrigan D R. Severe dysfunction of natural killer (NK) cells associated with chronic active human Herpesvirus-6 (HHV-6) viremia in patients with chronic fatigue syndrome. Abstract. IDSA. 37^{th} Annual Meeting. Nov. 18-21, 1999. Philadelphia, Pa.

17. Fudenberg H H and Pizza G. Transfer factor 1993: New frontiers. Progress in Drug Research 1994; 42: 311-400.

18. Wilson G B and Paddock G V. Process for obtaining transfer factor from colostrum, transfer factor so obtained and use thereof. 1989; U.S. Pat. No. 4,816,563.

19. Hana I, Vrubel J, Pekarek J and Cech K. The influence of age on transfer factor treatment of cellular immunodeficiency, chronic fatigue syndrome and/or chronic viral infections. Biotherapy 1996;9: 91-95.

20. De Vinci C, Levine P H, Pizza G et al. Lessons from a pilot study of transfer factor in chronic fatigue syndrome. Biotherapy 1996;9: 87-90.

21. Ablashi D V, Levine P H, De Vinci C et al. Use of HHV-6 transfer factor for the treatment of two patients with chronic fatigue syndrome (CFS). Two case reports. Biotherapy 1996;9: 81-86.

22. Rifkind R, Frey J A, Petersen E A and Dinowitz M. Transfer of delayed hypersensitivity in mice to microbial antigens with dialyzable transfer factor. Infec Immun 1977;16: 258-262.

23. Petersen EA, Greenberg LE, Manzara, T and Kirkpatrick CH. Murine transfer Factor. I. Description of the model and evidence for specificity. J Immunol 1981; 126: 2480-2484.

24. Wilson GB and Fudenberg HH. Use of in vitro assay techniques to measure parameters related to clinical applications of transfer therapy. 1986; U.S. Pat. No. 4,610,878.

25. Bryant J, Day R, Whiteside T L et al. Calculation of lytic units for the expression of Cell-mediated cytotoxicity. J Immunol Methods 1992; 146:91-103.

What is claimed is:

1. A transfer factor effective to confer cell-mediated immunity wherein the immune response is to Human Herpesvirus-6A and Human Herpesvirus-6B.

2. A transfer factor effective to confer cell-mediated immunity wherein the immune response is to Human Herpesvirus-6A or Human Herpesvirus-6B

3. A method of producing the transfer factor of claim 1 or 2 which comprises immunizing a lactating animal with Human Herpesvirus-6A and Human Herpesvirus-6B, recovering colostrum from the animal, and preparing the transfer factor from the colostrum.

4. The method of claim 3 wherein the animal is a bovid.

5. A method of producing the transfer factor of claim 1 or 2 which comprises immunizing an animal with Human Herpesvirus-6A and Human Herpesvirus-6B, recovering an immune system component from the animal, and preparing the transfer factor from the immune system component.

6. The method of claim 5 wherein the immune system component is dialyzable leukocyte extract.

7. The method of claim 5 wherein the immune system component is immune organ lysate.

8. The method of claim 5 wherein the immune system component is a cell line derived from an immune system component.

9. The method of claim 5 wherein the immune system component is a lymphoblastoid cell.

10. A method of producing a composition which comprises producing the transfer factor of claim 3 or 5 and admixing a carrier.

11. A method of producing a composition which comprises producing the transfer factor of claim 3 or 5 and admixing a carrier wherein the carrier is a pharmaceutically acceptable carrier.

12. A method of producing a composition which comprises producing the transfer factor of claim 3 or 5 and admixing a carrier wherein the carrier is an edible carrier.

13. A method of treating Chronic Fatigue Syndrome in a subject, which comprises administering to the subject an amount of Human Herpesvirus-6A and Human Herpesvirus-6B transfer factors effective to treat the Chronic Fatigue Syndrome.

14. A method of treating Multiple Sclerosis in a subject, which comprises administering to the subject an amount of Human Herpesvirus-6 transfer factor effective to treat the Multiple Sclerosis.

15. A method of treating Multiple Sclerosis in a subject, which comprises administering to the subject an amount of Human Herpesvirus-6A and Human Herpesvirus-6B transfer factors effective to treat the Multiple Sclerosis.

16. A method of treating an abnormality in a subject, which comprises administering to the subject an amount of the transfer factor of claim 2 effective to alleviate the abnormality, wherein the abnormality is alleviated by enhancing the subject's immune response to Human Herpesvirus-6A.

17. A method of treating an abnormality in a subject, which comprises administering to the subject an amount of the transfer factor of claim 2 effective to alleviate the abnormality, wherein the abnormality is alleviated by enhancing the subject's immune response to Human Herpesvirus-6B.

18. A method of treating an abnormality in a subject, which comprises administering to the subject an amount of the transfer factor of claim 1 effective to alleviate the abnormality, wherein the abnormality is alleviated by enhancing the subject's immune response to Human Herpesvirus-6A and Human Herpesvirus-6B.

19. A composition comprising the transfer factor of claim 1 or 2 and a carrier.

20. A composition comprising the transfer factor of claim 1 or 2 and a carrier, wherein the carrier is a pharmaceutically acceptable carrier.

21. A composition comprising the transfer factor of claim 1 or 2 and a carrier, wherein the transfer factor is present in an amount effective to enhance a subject's immune response.

22. A composition comprising the transfer factor of claim 1 or 2 and a carrier, wherein the carrier is a pharmaceutically acceptable carrier and wherein the transfer factor is present in an amount effective to enhance a subject's immune response.

23. A composition comprising the transfer factor of claim 1 or 2 and a carrier, wherein the carrier is an edible carrier and wherein the transfer factor is present in an amount effective to enhance a subject's immune response.

24. The composition of claim 19, wherein the transfer factor is capable of passing through a cell membrane.

25. Use of the transfer factor of claim 2 for the preparation of a pharmaceutical composition for treating an abnormality, wherein the abnormality is alleviated by enhancing the subject's immune response to Human Herpesvirus-6A.

26. Use of the transfer factor of claim 2 for the preparation of a pharmaceutical composition for treating an abnormality, wherein the abnormality is alleviated by enhancing the subject's immune response to Human Herpesvirus-6B.

27. Use of the transfer factor of claim 1 for the preparation of a pharmaceutical composition for treating an abnormality, wherein the abnormality is alleviated by enhancing the subject's immune response to Human Herpesvirus-6A and Human Herpesvirus-6B.

28. An edible composition comprising the transfer factor of claim 1 or 2 in an amount effective to enhance a person's immune response and an edible carrier.

29. A method of enhancing a person's immune response by administering to the person an amount of the composition of claim 28 effective to enhance a person's immune response.
